(19) United States
(12) Patent Application Publication
(10) Pub. No.: US 2019/0367568 A1
(43) Pub. Date: Dec. 5, 2019

(54) CALRETICULIN AND FUSION PROTEINS

(71) Applicants: NantBio, Inc., Culver City, CA (US); Nant Holding IP, LLC, Culver City, CA (US)

(72) Inventors: Patrick Soon-Shiong, Culver City, CA (US); Kayvan Niazi, Culver City, CA (US)

(21) Appl. No.: 16/266,724

(22) Filed: Feb. 4, 2019

Related U.S. Application Data
(60) Provisional application No. 62/626,551, filed on Feb. 5, 2018.

(51) Int. Cl. C07K 14/47; A61P 37/04; A61K 39/39; A61K 39/00

(52) U.S. Cl. CPC C07K 14/47; C07K 14/4748; A61P 37/04; A61K 39/39; C07K 2319/033; C07K 2319/03; C07K 2319/02; C07K 2319/04; A61K 2039/55594; A61K 39/0011

(57) ABSTRACT

Compositions, methods, and uses of recombinant calreticulin protein that is modified to be expressed on the cell surface are presented. Preferably, the recombinant calreticulin protein is presented on the antigen presenting cell surface with a tumor associated protein to increase immunogenicity of the tumor cell in the tumor microenvironment.

This application claims priority to our co-pending US provisional patent application with the Ser. No. 62/626,551, filed Feb. 5, 2018, which is incorporated by reference in its entirety herein.

### INCORPORATION BY REFERENCE

The instant application contains a Sequence Listing which has been submitted in ASCII format via EFS-Web and is hereby incorporated by reference in its entirety. Said ASCII copy was amended on Jun. 27, 2019, is named 102538-0060USAmendedSeqList.txt, and is 1,228 bytes in size.

### FIELD OF THE INVENTION

The field of the invention is immunotherapy, and especially as it relates to recombinant calreticulin peptides to increase immune response to tumor cells.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Calreticulin, also known as endoplasmic reticulum resident protein 60 (ERp60), is a typical ER residing protein with its ER retention sequence, KDEL (SEQ ID NO:1), which is located at the C-terminus of the protein and binds to KDEL receptor in the ER. Calreticulin is a multi-functional protein, which plays a role in preventing misfolded proteins from being exported from the ER, and also in chaperoning MHC I molecule to be prepared for binding an antigen for presentation on the cell surface.

More recently, attention has been drawn to the role of calreticulin as an “eat me” signal to nearby macrophages, which is recognized by the low density lipoprotein receptor-related protein (LRP). Calreticulin surface exposure is not a passive exposure of ER contents during cell death, but a highly regulated process of a preapoptotic event. For example, some cancer chemotherapy reagents (e.g., anthracyclines, etc.) induce the apoptosis-associated phosphatidylserine exposure, which is often preceded by calreticulin exposure in cancer cells. As calreticulin surface exposure on tumor cells elicits innate immune response (phagocytic attack on the tumor cell), calreticulin translocation is important for the immunogenicity of tumor cells as well as for effectiveness of cancer therapy.

Some tried to use such “eat me” signal of calreticulin to increase immunogenicity of the tumor cells. For example, U.S. Pat. Pub. No. 2016/0318975 to Baileykobayashi discloses synthetic peptides (e.g., siRNA-associated sequences of centrin 2 protein, or cytoskeleton-associated protein 5, etc.) to promote calreticulin expression. In another example, international application WO2016/118754, Weissman discloses increase of calreticulin surface expression on the phagocytic cells by treating TLR agonists or CD47 blocking reagents. In still other examples, U.S. Pat. Pub. No. 2009/0005302, Obeid teaches expression of recombinant (mimetic) calreticulin to trigger phagocytosis of a cancer cell. However, none of those effectively increase surface expression of calreticulin along in the context of tumor associated antigens.

Therefore, even though the relationship between immunogenicity and surface expression of calreticulin in tumor cells is known, it remains largely unexplored how to effectively increase the surface expression of calreticulin along with presentation of tumor associated antigen and to use such approach in a cancer vaccine.

Consequently, there is still a need for improved compositions, methods for and uses of recombinant calreticulin expressed on a cell surface, especially in context with tumor antigens.

### SUMMARY OF THE INVENTION

The inventive subject matter is directed to various compositions of, methods for, and use of recombinant calreticulin expressed on the cell surface in the context of tumor antigens. Thus, one aspect of the subject matter includes a recombinant nucleic acid having a plurality of nucleic acid segments. Typically the recombinant nucleic acid includes a first nucleic acid segment encoding at least a portion of calreticulin and a second nucleic acid segment encoding at least a portion of a transmembrane domain. Preferably, the first and second nucleic acid segments are present in a same reading frame. In some embodiments, the portion of the calreticulin is a mutant form of calreticulin, in which KDEL sequence is deleted from carboxyl terminus. Additionally, it is contemplated that at least a portion of the calreticulin is coupled with a membrane targeting signal sequence (e.g., C2 domain, etc.). In some embodiments, the second nucleic acid segment is coupled with the first nucleic acid segment via a linker. In such embodiments, the linker may comprise glycine-rich sequences. Optionally, the recombinant nucleic acid may further comprise a third nucleic acid segment encoding a tumor-associated antigen.

In another aspect of the inventive subject matter, the inventors also contemplate a recombinant expression vector for immune therapy. The recombinant expression vector includes a nucleic acid sequence that encodes a recombinant protein. The nucleic acid sequence has a plurality of nucleic acid segments, which typically includes a first nucleic acid segment encoding at least a portion of calreticulin and a second nucleic acid segment encoding at least a portion of a transmembrane domain. Preferably, the first and second nucleic acid segments are present in a same reading frame to form a hybrid protein having a calreticulin portion and a transmembrane domain portion. Optionally, the recombinant nucleic acid may further comprise a third nucleic acid segment encoding a tumor-associated antigen.

Typically, the expression vector can be selected from a group consisting of a viral expression vector, a bacterial expression vector, and a yeast expression vector. The viral expression vector may be an adenoviral expression vector having E1 and E2b genes deleted. The bacteria expression vector may be expressable in a genetically-engineered bacterium expresses endotoxins at a low level, which is insufficient to induce a CD-14 mediated sepsis. The yeast expression vector may be expressable in S. cerevisiae.

In some embodiments, the portion of the calreticulin is a mutant form of calreticulin, in which KDEL sequence is deleted from carboxyl terminus. In some embodiments, the second nucleic acid segment is coupled with the first nucleic acid segment via a linker, which can comprise glycine-rich sequences. In some embodiments, the portion of the calreticulin is coupled with a membrane targeting signal sequence, which can be C2 domain.

Still another aspect of inventive subject matter is directed towards a recombinant nucleic acid having plurality of nucleic acid segments. Typically the recombinant nucleic acid includes a first nucleic acid segment encoding at least a portion of calreticulin and a second nucleic acid segment encoding a tumor associated antigen. Preferably, the first and second nucleic acid segments are present in a same reading frame. In some embodiments, the portion of the calreticulin is a mutant form of calreticulin, in which KDEL sequence is deleted from carboxyl terminus. In some embodiments, the second nucleic acid segment is coupled with the first nucleic acid segment via a linker, which can comprise glycine-rich sequences. In some embodiments, the portion of the calreticulin is coupled with a membrane targeting signal sequence, which can be C2 domain.

It is contemplated that in some embodiments, the tumor associated antigen is a patient- and tumor-specific neoepitope. In such embodiments, it is preferred that the neoepitope is filtered to have binding affinity to an MHC-I or MHC-II complex of equal or less than 500 nM and/or filtered against known human SNP and somatic variations. In some embodiments, the second nucleic acid segment is coupled with the first nucleic acid segment via a linker, which can comprise glycine-rich sequences.

In still another aspect of the inventive subject matter, the inventors further contemplate a recombinant expression vector for immune therapy. The recombinant expression vector includes a nucleic acid sequence that encodes a recombinant protein. The nucleic acid sequence has a plurality of nucleic acid segments, which typically includes a first nucleic acid segment encoding at least a portion of calreticulin and a second nucleic acid segment encoding a tumor associated antigen. Preferably, the first and second nucleic acid segments are present in a same reading frame to either form a hybrid protein or two separate translation units (e.g., separated by a P2A sequence). In some embodiments, the portion of the calreticulin is a mutant form of calreticulin, in which KDEL sequence is deleted from carboxyl terminus. It is contemplated that in some embodiments, the tumor associated antigen is a patient- and tumor-specific neoepitope. In such embodiments, it is preferred that the neoepitope is filtered to have binding affinity to an MHC-I or MHC-II complex of equal or less than 500 nM and/or filtered against known human SNP and somatic variations.

Typically, the expression vector can be selected from a group consisting of a viral expression vector, a bacterial expression vector, and a yeast expression vector. The viral expression vector may be an adenoviral expression vector having E1 and E2b genes deleted. The bacteria expression vector may be expressable in a genetically-engineered bacterium expresses endotoxins at a low level, which is insufficient to induce a CD-14 mediated sepsis. The yeast expression vector may be expressable in S. cerevisiae.

In still another aspect of the inventive subject matter, the inventors contemplate a method of increasing effectiveness of immune therapy to a patient having a tumor. In this method a pharmaceutical composition comprising a nucleic acid sequence that encodes a recombinant protein is provided. Typically the recombinant nucleic acid includes a first nucleic acid segment encoding at least a portion of calreticulin and a second nucleic acid segment encoding at least a portion of a transmembrane domain. Preferably, the first and second nucleic acid segments are present in a same reading frame. Then the pharmaceutical composition is administered to the patient in a dose and schedule effective to treat the tumor. In some embodiments, the portion of the calreticulin is a mutant form of calreticulin, in which KDEL sequence is deleted from carboxyl terminus. In other embodiments, the portion of the calreticulin is coupled with a membrane targeting signal sequence (e.g., C2 domain, etc.). In some embodiments, the second nucleic acid segment is coupled with the first nucleic acid segment via a linker. In such embodiments, the linker may comprise glycine-rich sequences. It is contemplated that at least a portion of calreticulin is exposed on the plasma membrane of a tumor cell when the pharmaceutical composition is administered to the patient.

Most typically, the pharmaceutical composition is selected from a group consisting of a viral vaccine, a bacteria vaccine, a yeast vaccine. Optionally, the pharmaceutical composition further comprises a nucleic acid sequence that encodes a tumor-associated antigen. In such embodiment, it is preferred that the nucleic acid sequence that encodes a recombinant protein and the nucleic acid sequence that encodes a tumor-associated antigen generate two distinct peptides.

In still another aspect of the inventive subject matter, the inventors contemplate a method of increasing effectiveness of immune therapy to a patient having a tumor. In this method a pharmaceutical composition comprising a nucleic acid sequence that encodes a recombinant protein is provided. Typically the recombinant nucleic acid includes a first nucleic acid segment encoding at least a portion of calreticulin and a second nucleic acid segment encoding a tumor associated antigen. Preferably, the first and second nucleic acid segments are present in a same reading frame. Then the pharmaceutical composition is administered to the patient in a dose and schedule effective to treat the tumor. Typically, the expression vector can be selected from a group consisting of a viral expression vector, a bacterial expression vector, and a yeast expression vector. In some embodiments, the portion of the calreticulin is a mutant form of calreticulin, in which KDEL sequence is deleted from carboxyl terminus. In some embodiments, the portion of the calreticulin is a mutant form of calreticulin, in which KDEL sequence is deleted from carboxyl terminus.

It is contemplated that the tumor associated antigen is a patient- and tumor-specific neoepitope. In such embodiments, it is preferred that the neoepitope is filtered to have binding affinity to an MHC-I or MHC-II complex of equal or less than 500 nM and/or filtered against known human SNP and somatic variations. In some embodiment, the tumor associated antigen is a polytope, and/or the polytope comprises a plurality of filtered neoepitope peptides.

It is contemplated that at least a portion of calreticulin is exposed on the plasma membrane of a tumor cell when the pharmaceutical composition is administered to the patient. Most typically, the pharmaceutical composition is selected from a group consisting of a viral vaccine, a bacteria vaccine, a yeast vaccine.

In still another aspect of the inventive subject matter, the inventors contemplate a pharmaceutical composition that includes a recombinant peptide and an adjuvant molecule stimulating a dendritic cell. The recombinant peptide comprises at least a portion of calreticulin and a tumor associated antigen, and preferably associated with a pharmaceutically acceptable molecular carrier. In some embodiments, the adjuvant molecule includes Bacillus Calmette-Guerin (BCG, preferably inactivated) to promote uptake of the recombinant peptide into the dendritic cells. It is contemplated that, in some embodiments, the tumor associated antigen is a patient- and tumor-specific neoepitope and/or a polytope. In such embodiments, it is preferred that the neoepitope is filtered to have binding affinity to an MHC-I or MHC-II complex of equal or less than 500 nM and/or filtered against known human SNP and somatic variations.

Still another aspect of the inventive subject matter includes a method of increasing effectiveness of immune therapy to a patient having a tumor. In this method, a pharmaceutical composition that includes a recombinant peptide and an adjuvant molecule stimulating a dendritic cell is provided. The recombinant peptide comprises at least a portion of calreticulin and a tumor associated antigen, and preferably associated with a pharmaceutically acceptable molecular carrier. Then, the pharmaceutical composition is administered to the patient in a dose and schedule effective to treat the tumor. In some embodiments, the adjuvant molecule includes Bacillus Calmette-Guerin (BCG, preferably inactivated) to promote uptake of the recombinant peptide into the dendritic cells. It is contemplated that, in some embodiments, the tumor associated antigen is a patient- and tumor-specific neoepitope and/or a polytope. In such embodiments, it is preferred that the neoepitope is filtered to have binding affinity to an MHC-I or MHC-II complex of equal or less than 500 nM and/or filtered against known human SNP and somatic variations.

It is contemplated that at least a portion of calreticulin is exposed on the plasma membrane of a tumor cell when the pharmaceutical composition is administered to the patient. Most typically, the pharmaceutical composition is selected from a group consisting of a viral vaccine, a bacteria vaccine, a yeast vaccine.

In still another aspect of the inventive subject matter, the inventors contemplate use of recombinant nucleic acids, expression vectors, or pharmaceutical compositions described above described above for increasing effectiveness of immune therapy to a patient having a tumor.

Various objects, features, aspects and advantages of the inventive subject matter will become more apparent from the following detailed description of preferred embodiments.

### DETAILED DESCRIPTION

The inventors now discovered that immune therapy, and especially neoepitope-based immune therapy can be further improved by triggering or increasing surface expression of calreticulin on the cell membrane of antigen presenting cells along with presentation of tumor associated antigen. Such increased surface expression of calreticulin (with tumor associated antigen) can be achieved by delivering recombinant nucleic acid or recombinant protein including calreticulin fragment that is modified to be preferentially trafficked to the cell surface to the antigen presenting cells in the tumor microenvironment. The antigen presenting cells that express the recombinant protein or uptake the recombinant protein are likely to present at least a portion of calreticulin along with tumor associate antigens in its vicinity on the cell surface, by which immunogenicity of the antigen presenting cells may be substantially elicited and/or increased.

To that end, the inventors discovered that various recombinant nucleic acid compositions or vaccine compositions can be generated to modify the antigen presenting cells (e.g., dendritic cells, etc.) such that the antigen presenting cells can present calreticulin and tumor associated antigen on the cell surface. In one exemplary and especially preferred aspect of the inventive subject matter, the inventors contemplate that antigen presenting cells of a patient can be modified to present a recombinant calreticulin peptide on the cell surface by introducing a recombinant nucleic acid composition encoding the recombinant protein. Generally, the recombinant protein includes at least a portion of calreticulin protein associated with a peptide that can trigger surface expression of the recombinant protein. Thus, in a preferred embodiment, in which the recombinant protein is encoded by a single recombinant nucleic acid, the recombinant nucleic acid includes at least two nucleic acid segments: a first nucleic acid segment (a sequence element) encoding at least a portion of calreticulin; and a second nucleic acid segment (a sequence element) encoding a peptide triggering surface expression of the recombinant protein. Most preferably, the two nucleic acid segments are in the same reading frame such that two nucleic acid segments can be translated into a single protein having two peptide segments.

As used herein, the term “tumor” refers to, and is interchangeably used with one or more cancer cells, cancer tissues, malignant tumor cells, or malignant tumor tissue, that can be placed or found in one or more anatomical locations in a human body. As used herein, the term “bind” refers to, and can be interchangeably used with a term “recognize” and/or “detect”, an interaction between two molecules with a high affinity with a K_{D} of equal or less than 10⁻⁶M, or equal or less than 10⁻⁷M. As used herein, the term “provide” or “providing” refers to and includes any acts of manufacturing, generating, placing, enabling to use, or making ready to use.

It is contemplated any portion of calreticulin that can be recognized as a “eat me” signal to the phagocytes to so elicit immune response against the cell expressing such portion of calreticulin or to so render the cells expressing such portion of calreticulin more immunogenic can be used. Thus, the portion of calreticulin may include at least 20%, at least 30%, at least 50%, at least 70%, at least 90% of the wildtype calreticulin. In addition, while the portion of calreticulin can include any part of calreticulin or any combination thereof, it is preferred that the portion of calreticulin includes at least 30 amino acids, preferably at least 50 amino acids, more preferably at least 100 amino acids from the N-terminus of the wildtype calreticulin. Thus, in some embodiments, the portion of calreticulin may include mixed sequences of calreticulin from different portions of the calreticulin. For example, where the portion of calreticulin comprises 50% of the wildtype calreticulin sequences, the portion of calreticulin may comprise a half of wild type calreticulin from N-terminus as a continuous sequence (first 200 amino acid sequences of calreticulin), or alternatively, a 50 amino acids from N-terminus (e.g., amino acid (aa) 1-50, aa 50-100, aa 100-150, aa 150-200, etc.) fused with other part of the calreticulin (e.g., aa 200-350, aa 250-400, etc.).

Alternatively and/or preferably, the portion of calreticulin is a fragment of calreticulin that lacks ER retention signal sequence of calreticulin (KDEL in C terminus) or a mutant calreticulin, in which ER retention signal sequence is substituted with other amino acid sequences of the same length or different length (e.g., with a peptide sequence of GGGG (SEQ ID NO:2), GGGGGG (SEQ ID NO:3), GGEL (SEQ ID NO:4), GDGL (SEQ ID NO:5), etc.). In such embodiment, the deletion, substitution and/or lack of ER retention signal sequence may release calreticulin peptide from ER such that calreticulin is more likely to be transported to the cell membrane. However, it is also contemplated that the portion of calreticulin retains a full or a portion of ER retention signal sequence if the peptide triggering surface expression is coupled to the C-terminus of the portion of calreticulin (i.e., adjacent to the KDEL (SEQ ID NO:1) sequence) such that the peptide triggering surface expression can provide a steric hindrance to the KDEL receptor to bind to the KDEL sequence.

Alternatively or additionally, the portion of calreticulin can be modified to include a membrane targeting signal sequence with or without deletion of KDEL sequence. The membrane targeting signal sequence can be placed in N terminus, C terminus, or can be embedded in any portion of calreticulin depending on the type of membrane targeting signal sequences. For example, the membrane targeting signal sequence can be C1 domain (e.g., protein kinase C (PKC) conserved 1 (C1), etc.), or C2 domain (e.g., protein kinase C (PKC) conserved 2 (C1), etc.), or pleckstrin homology domains, which can be preferentially located at the N terminus of the portion of calreticulin. In another example, the membrane targeting signal sequence can be a B. subtilis MinD membrane targeting sequence or a bacterial actin homologue FtsA membrane targeting sequence, which can be preferentially located at the C terminus of the portion of calreticulin.

Such obtained or generated calreticulin portion can be further combined (e.g., fused, linked, etc.) with a peptide that can trigger surface expression of the calreticulin portion on the cell membrane. Any suitable peptides that can trigger surface expression of the protein are contemplated. For example, in one preferred embodiment, the suitable peptide includes a transmembrane domain, with which the portion of calreticulin protein can be anchored on the plasma membrane to so be presented on the cell surface. Thus, suitable transmembrane domains will have a length (e.g., single transmembrane domain, double transmembrane domain, triple transmembrane domain, etc.) that will not trigger misfolding of the recombinant protein or instability of RNA transcript. An exemplary transmembrane domain may include a transmembrane domain of an immunoglobulin, of a T cell receptor, or of a MHC I/II molecule. While any suitable configuration is contemplated, it is especially preferred that the transmembrane domain is coupled with the portion of calreticulin at the C terminus of calreticulin such that most of the portion of calreticulin can be exposed extracellularly on the cell surface. However, it is also contemplated that, where the transmembrane domain includes a plurality of transmembrane domain (e.g., double or triple transmembrane domain or more, etc.), the portion of calreticulin or its fragments, or different portion of calreticulin can be placed in between the transmembrane domain such that a plurality portions of calreticulin can be exposed extracellularly on the cell surface.

Optionally, the recombinant nucleic acid may include a third nucleic acid segment encoding a tumor associated antigen or portions thereof such that when the recombinant nucleic acid is transfected into a cell, the recombinant calreticulin peptide and the tumor associated antigen or portions thereof can be co-expressed in the same cell. In some embodiments, the third nucleic acid segment may be placed in the same reading frame with the first and second nucleic acid fragments. In other embodiments, the third nucleic acid segment may be placed in different reading frame (under a different promoter) from the first and second nucleic acid fragment. Alternatively, first/second nucleic acid segments and the third nucleic acid segment may be coupled via an IRES element. In any embodiment, it is preferred that first and second nucleic acid fragments and the third nucleic acid fragment encode the recombinant calreticulin peptide and the tumor associated protein as two distinct and separate peptide (not linked or fused with each other) such that the recombinant calreticulin are trafficked to the cell surface via the surface targeting domain (a peptide that can trigger surface expression of the calreticulin portion on the cell membrane) and the tumor associated protein can be processed intracellularly to be presented with MHC molecule (by being associated with MHC I or MHC II molecule).

Alternatively, the suitable peptide may include a tumor associated antigen, with which the portion of calreticulin protein can be processed to be present on the cell surface by being associated with MHC I or MHC II molecule. Without wishing to be bound to any specific theory, it is contemplated that calreticulin associated with the tumor associated antigen will be processed together to generate MHC II-antigen complex by bypassing the ER retention mechanism. In some embodiments, a fragment of calreticulin can be associated with a fragment of tumor associated antigen to so generate a hybrid antigen (a fused peptide) to be bound to MHC II molecule. In other embodiments, a fragment of calreticulin can be independently associated with an MHC II molecule to so generate a distinct MHC-antigen complex from MHC-tumor associated antigen complex. In such embodiments, it is contemplated that the calreticulin protein can be processed intracellularly such that at least a portion will be transported to the cell membrane.

In some embodiments, the tumor associated antigen is a tumor-specific, patient-specific neoepitope. As used herein, the tumor-associated antigen refers any antigen that can be presented on the surface of the tumor cells, which includes an inflammation-associated peptide antigen, a tumor associated peptide antigen, a tumor specific peptide antigen, and a cancer neoepitope. Typically, the tumor associated antigens and neoepitopes (which are typically patient-specific and tumor-specific) can be identified from the omics data obtained from the cancer tissue of the patient or normal tissue (of the patient or a healthy individual), respectively. Omics data of tumor and/or normal cells preferably comprise a genomic data set that includes genomic sequence information. Most typically, the genomic sequence information comprises DNA sequence information that is obtained from the patient (e.g., via tumor biopsy), most preferably from the tumor tissue (diseased tissue) and matched healthy tissue of the patient or a healthy individual. For example, the DNA sequence information can be obtained from a pancreatic cancer cell in the patient's pancreas (and/or nearby areas for metastasized cells), and a normal pancreatic cells (non-cancerous cells) of the patient or a normal pancreatic cells from a healthy individual other than the patient.

In one especially preferred aspect of the inventive subject matter, DNA analysis is performed by whole genome sequencing and/or exome sequencing (typically at a coverage depth of at least 10×, more typically at least 20×) of both tumor and matched normal sample. Alternatively, DNA data may also be provided from an already established sequence record (e.g., SAM, BAM, FASTA, FASTQ, or VCF file) from a prior sequence determination. Therefore, data sets may include unprocessed or processed data sets, and exemplary data sets include those having BAM format, SAM format, FASTQ format, or FASTA format. However, it is especially preferred that the data sets are provided in BAM format or as BAMBAM diff objects (see e.g., US2012/0059670A1 and US2012/0066001A1). Moreover, it should be noted that the data sets are reflective of a tumor and a matched normal sample of the same patient to so obtain patient and tumor specific information. Thus, genetic germ line alterations not giving rise to the tumor (e.g., silent mutation, SNP, etc.) can be excluded such that the neoepitope is filtered against known human SNP and somatic variations. Of course, it should be recognized that the tumor sample may be from an initial tumor, from the tumor upon start of treatment, from a recurrent tumor or metastatic site, etc. In most cases, the matched normal sample of the patient may be blood, or non-diseased tissue from the same tissue type as the tumor.

The so obtained neoepitopes may then be subject to further detailed analysis and filtering using predefined structural and expression parameters, and sub-cellular location parameters. For example, it should be appreciated that neoepitope sequences are only retained provided they will meet a predefined expression threshold (e.g., at least 20%, 30%, 40%, 50%, or higher expression as compared to normal) and are identified as having a membrane associated location (e.g., are located at the outside of a cell membrane of a cell). Further contemplated analyses will include structural calculations that delineate whether or not a neoepitope or a tumor associated antigen, or a self-lipid is likely to be solvent exposed, presents a structurally stable epitope, etc.

Consequently, it should be recognized that epitopes can be identified in an exclusively in silico environment that ultimately predicts potential epitopes that are unique to the patient and tumor type. So identified epitope sequences are then synthesized in vitro to generate the corresponding peptides. Thus, it is conceptually possible to assemble an entire rational-designed collection of (neo)epitopes of a specific patient with a specific cancer, which can then be further tested in vitro to find or generate high-affinity antibodies. In one aspect of the inventive subject matter, one or more of the peptide (neo)epitopes (e.g., 9-mers) can be immobilized on a solid carrier (e.g., magnetic or color coded bead) and used as a bait to bind surface presented antibody fragments or antibodies. Most typically, such surface presented antibody fragments or antibodies are associated with a M13 phage (e.g., protein III, VIII, etc.) and numerous libraries for antibody fragments are known in the art and suitable in conjunction with the teachings presented herein. Where desired, smaller libraries may also be used and be subjected to affinity maturation to improve binding affinity (e.g., binding affinity to an MHC-I or MHC-II complex of equal or less than 500 nM, equal or less than 200 nM, etc.) and/or kinetic using methods well known in the art (see e.g., Briefings in functional genomics and proteomics. Vol 1. No 2.189-203. July 2002). In addition, it should be noted that while antibody libraries are generally preferred, other scaffolds are also deemed suitable and include beta barrels, ribosome display, cell surface display, etc. (see e.g., Protein Sci. 2006 January; 15(1): 14-27.) In addition, as already discussed above, it should be appreciated that not only patient and tumor specific neoepitopes are deemed suitable, but also all known tumor associated antigens (e.g., CEACAM, MUC-1, HER2, etc.).

In some embodiments, the tumor associated antigen can be a polytope. As used herein, a polytope refers a tandem array of two or more antigens (or neoepitopes) expressed as a single polypeptide. Preferably, two or more human disease-related antigens are separated by a linker or spacer peptides. Any suitable length and order of peptide sequence for the linker or the spacer can be used. However, it is preferred that the length of the linker peptide is between 3-30 amino acids, preferably between 5-20 amino acids, more preferably between 5-15 amino acids. Also inventors contemplates that glycine-rich sequences (e.g., gly-gly-ser-gly-gly, (SEQ ID NO: 6) etc.) are preferred to provide flexibility of the polytope between two antigens.

Optionally, the portion of calreticulin and the peptide triggering surface expression of the recombinant protein can be coupled via a linker. Any suitable length and order of peptide sequence for the linker or the spacer can be used and the suitable length of the linker may vary depending on the type and sequence of the portion of calreticulin and the peptide. Generally, it is preferred that the length of the linker peptide is between 3-30 amino acids, preferably between 5-20 amino acids, more preferably between 5-15 amino acids. Also inventors contemplates that glycine-rich sequences (e.g., gly-gly-ser-gly-gly, (SEQ ID NO: 6) etc.) are preferred to provide flexibility of the polytope between two antigens. In addition, where the portion of calreticulin includes the ER retention sequence (KDEL (SEQ ID NO:1)) in its C terminus, it is preferred that the length of the linker peptide is short such that the KDEL sequence is not fully exposed and recognizable by the KDEL receptor. Thus, the preferred length in such embodiment is between 3-15 amino acids, preferably between 3-10 amino acids.

It is contemplated that such generated recombinant nucleic acids (e.g., nucleic acid encoding calreticulin fused with a transmembrane domain, calreticulin fused with a tumor associated antigen) can be further inserted into an expression vector such that recombinant peptide can be expressed by a genetically-engineered microorganism (e.g., virus, bacteria or yeast, etc.). A recombinant nucleic acid encoding the recombinant protein (e.g., calreticulin fused with a transmembrane domain, calreticulin fused with a tumor associated antigen, etc.) can be placed in an expression vector such that nucleic acid encoding the recombinant protein can be delivered to an antigen presenting cell (e.g., dendritic cells, etc.) of the patient, or to transcribe the nucleic acid sequence in bacteria or yeast so that the recombinant protein encoded by the nucleic acid sequence can be, as a whole, or as fragments, delivered to the antigen presenting cell. Any suitable expression vectors that can be used to express protein are contemplated. Especially preferred expression vectors may include those that can carry a cassette size of at least 1 k, preferably 2 k, more preferably 5 k base pairs.

Thus, in one embodiment, the microorganism is a virus, and a preferred expression vector includes a viral vector that may be derived from any suitable virus including adenoviruses, adeno-associated viruses, alphaviruses, herpes viruses, lentiviruses, etc. However, adenoviruses are particularly preferred. Moreover, it is further preferred that the virus is a replication deficient and non-immunogenic virus, which is typically accomplished by targeted deletion of selected viral proteins (e.g., E1, E3 proteins). Such desirable properties may be further enhanced by deleting E2b gene function, and high titers of recombinant viruses can be achieved using genetically modified human 293 cells as has been recently reported (e.g., J Virol. 1998 February; 72(2): 926-933). Thus, the inventors contemplate that one desired viral vector may include a recombinant adenovirus genome with a deleted or non-functional E2b gene.

In still further embodiments, the microorganism is a bacteria, and the expression vector can be a bacterial vector that can be expressed in a genetically-engineered bacterium, which expresses endotoxins at a level low enough not to cause an endotoxic response in human cells and/or insufficient to induce a CD-14 mediated sepsis when introduced to the human body. One exemplary bacteria strain with modified lipopolysaccharides includes ClearColi® BL21(DE3) electrocompetent cells. This bacteria strain is BL21 with a genotype F- ompT hsdSB (rB- mB) gal dcm lon κ(DE3 [lacI lacUV5-T7 gene 1 ind1 sam7 nin5]) msbA148 ΔgutQΔkdsD ΔlpxLΔlpxMΔpagPΔlpxPΔeptA. In this context, it should be appreciated that several specific deletion mutations (ΔgutQ ΔkdsD ΔlpxL ΔlpxMΔpagPΔlpxPΔeptA) encode the modification of LPS to Lipid IV_{A}, while one additional compensating mutation (msbA148) enables the cells to maintain viability in the presence of the LPS precursor lipid IVA. These mutations result in the deletion of the oligosaccharide chain from the LPS. More specifically, two of the six acyl chains are deleted. The six acyl chains of the LPS are the trigger which is recognized by the Toll-like receptor 4 (TLR4) in complex with myeloid differentiation factor 2 (MD-2), causing activation of NF-kB and production of proinflammatory cytokines. Lipid IV_{A}, which contains only four acyl chains, is not recognized by TLR4 and thus does not trigger the endotoxic response. While electrocompetent BL21 bacteria is provided as an example, the inventors contemplates that the genetically modified bacteria can be also chemically competent bacteria. Alternatively, or additionally, the microorganism is yeast, and the expression vector can also be a yeast vector that can be expressed in yeast, preferably, in Saccharomyces cerevisiae (e.g., GI-400 series recombinant immunotherapeutic yeast strains, etc.)

The inventors further contemplated that the recombinant virus, bacteria or yeast having recombinant nucleic acid as described above can be further formulated in any pharmaceutically acceptable carrier (e.g., preferably formulated as a sterile injectable composition) to form a pharmaceutical vaccine composition (virus vaccine, bacteria vaccine, and/or yeast vaccine). Where the pharmaceutical composition includes the recombinant virus, it is preferred that a virus titer of the composition is between 10⁴-10′² virus particles per dosage unit. However, alternative formulations are also deemed suitable for use herein, and all known routes and modes of administration are contemplated herein. Where the pharmaceutical composition includes the recombinant bacteria, it is preferred that the bacteria titer of the composition 10²-10³, 10³-10⁴, 10⁴-10⁵ bacteria cells per dosage unit. Where the pharmaceutical composition includes the recombinant yeast, it is preferred that the bacteria titer of the composition 10²-10³, 10³-10⁴, 10⁴-10⁵ yeast cells per dosage unit.

The inventors also contemplate that the recombinant protein can be expressed in vitro by transforming peptide producing bacteria (e.g., BL-21, etc.) and further isolated and purified. Such purified recombinant protein can then be associated with a pharmaceutically acceptable carrier such that the recombinant protein can be directly delivered to the tumor microenvironment. Any pharmaceutically acceptable carrier (e.g., preferably formulated as a sterile injectable composition) that can stably carry the recombinant proteins to the tumor microenvironment are contemplated. One exemplary carrier includes a nano particle to which the recombinant proteins can be directly or indirectly linked. The nano particle can be a bead, a nanoparticle, or a protein molecule that can be conjugated (or linked) with the recombinant peptide and the (synthetic) glycolipid. For example, the nano particle may include, but not limited to, protein A, protein G, protein Z, albumin, and refolded albumin. Especially, where the carrier protein is an albumin, the a hydrophobic recombinant peptide and/or (synthetic) glycolipids may fit in one of Sudlow's site I and II of the albumin or any other hydrophobic area of the albumin. In some embodiments where the recombinant peptide is not hydrophobic enough, it is contemplated that the recombinant peptide can be coupled with an hydrophobic short anchor peptide (in a length of at least 10 amino acids, 15 amino acids, 20 amino acids, 30 amino acids, etc.) such that the recombinant peptide can be placed at the Sudlow's site I and II of the albumin via the hydrophobic short anchor peptide.

Optionally, in some embodiments, the recombinant protein may further be associated with a dendritic cell targeting moiety to increase the specificity and effectiveness of the recombinant protein. The inventors contemplate that the recombinant protein can be specifically targeted to the dendritic cells using a binding molecule to a mannose receptor (e.g., CD206, etc.), which is a hallmark molecule for immature dendritic cells. While any suitable binding molecules that can specifically recognize at least a portion of the mannose receptor (preferably human mannose receptor) are contemplated, a preferred binding molecule includes a mannose-derived polysaccharide (e.g., mannose-dextran, mannan, lipoarabinomannan, etc.), fucose-derived/containing polysaccharide, or N-acetylglucosamine-derived/containing polysaccharide, or any other mannose receptor interacting molecules (e.g., agalactosyl IgG, etc.), which may facilitate uptake of the recombinant protein into the dendritic cell upon binding to the mannose receptor.

It is contemplated that such prepared expression vectors or vaccines (e.g., virus, bacteria, yeast) or the recombinant protein associated with a carrier can be administered to the patient in a dose and effective to treat the tumor. As used herein, the term “administering” a virus, bacterial or yeast formulation, or the recombinant protein associated with a carrier refers to both direct and indirect administration of those formulations. Direct administration of the formulation is typically performed by a health care professional (e.g., physician, nurse, etc.), and indirect administration includes a step of providing or making available the formulation to the health care professional for direct administration (e.g., via injection, infusion, oral delivery, topical delivery, etc.). In some embodiments, the virus, bacterial or yeast formulation is administered via systemic injection including subcutaneous, subdermal injection, or intravenous injection. In other embodiments, where the systemic injection may not be efficient (e.g., for brain tumors, etc.) or less therapeutically effective, it is contemplated that the formulation is administered via intratumoral injection.

With respect to dose and schedule of the formulation administration, it is contemplated that the dose and/or schedule may vary depending on depending on the type of virus, bacteria or yeast, type and prognosis of disease (e.g., tumor type, size, location), health status of the patient (e.g., including age, gender, etc.). While it may vary, the dose and schedule may be selected and regulated so that the formulation does not provide any significant toxic effect to the host normal cells, yet sufficient to be elicit cytotoxic immune cell-mediated immune response. Thus, in a preferred embodiment, an optimal or desired condition of administering the formulation can be determined based on a predetermined threshold. For example, the predetermined threshold may be a predetermined local or systemic concentration of specific type of cytokine (e.g., IFN-γ, TNF-β, IL-2, IL-4, IL-10, etc.). Therefore, administration conditions are typically adjusted to have NKT- or NK-specific cytokines released or expressed at least 20%, at least 30%, at least 50%, at least 60%, at least 70% more than untreated conditions (e.g., in the same patient before the treatment or different patient with similar conditions without treatment, etc.), at least locally or systemically.

For example, where the pharmaceutical composition includes the recombinant virus, the contemplated dose of the oncolytic virus formulation is at least 10⁶ virus particles/day, or at least 10⁸ virus particles/day, or at least 10¹⁰ virus particles/day, or at least 10¹¹ virus particles/day. In some embodiments, a single dose of virus formulation can be administered at least once a day or twice a day (half dose per administration) for at least a day, at least 3 days, at least a week, at least 2 weeks, at least a month, or any other desired schedule. In other embodiments, the dose of the virus formulation can be gradually increased during the schedule, or gradually decreased during the schedule. In still other embodiments, several series of administration of virus formulation can be separated by an interval (e.g., one administration each for 3 consecutive days and one administration each for another 3 consecutive days with an interval of 7 days, etc.).

In some embodiments, the administration of the pharmaceutical formulation can be in two or more different stages: a priming administration and a boost administration. It is contemplated that the dose of the priming administration is higher than the following boost administrations (e.g., at least 20%, preferably at least 40%, more preferably at least 60%). Yet, it is also contemplated that the dose for priming administration is lower than the following boost administrations. Additionally, where there is a plurality of boost administration, each boost administration has different dose (e.g., increasing dose, decreasing dose, etc.).

Without wishing to be bound by any specific theory, the inventors contemplate that administration of pharmaceutical vaccine composition (e.g., as a recombinant viral, bacterial, or yeast composition) to a patient will cause the antigen presenting cells in the patient (e.g., especially dendritic cells) to process the recombinant protein to so present calreticulin or its fragment thereof as antigens coupled with MHC protein on the surface. It is expected that co-presentation the calreticulin and the tumor associated antigen (preferably neoepitope) on the antigen presenting cell will elicit and/or increase the immunogenicity of the tumor cells to so induce further or boosted immune responses against the tumor cell.

The inventors also contemplate that the pharmaceutical composition of the recombinant protein associated with a carrier can be administered directly to the patient with an adjuvant that can stimulate the antigen presenting cells. Any suitable adjuvants that can stimulate the antigen presenting cells to increase the uptake of the extracellular antigen without producing significant toxic side effects to the immune system are contemplated. Exemplary adjuvants may include Bacille Calmette-Guerin (BCG) that can activate the antigen presenting cells by activating toll-like receptors (TLRs). As BCG carries risk of systemic mycobacterial infection, it is preferred that the BCG is inactivated (e.g., by heat, sonication, irradiation, etc.) before administration. Other adjuvants may include TLR agonists (e.g., TLR3 agonist poly-ICLC, or a TLR9 agonist such as synthetic oligonucleotides containing CpG motifs, etc.) that can be optionally coadministered with a long (20-mer) peptide in IFA (incomplete Freund's adjuvant), cytokines (e.g., IL-12, GM-CSF, etc.) and low dose cyclophosphamide. In some embodiments, the adjuvant can be co-administered to the patient with the recombinant protein associated with a carrier. In other embodiments, the adjuvant can be administered (e.g., at least once, twice, in a pulse, etc.) before administering the recombinant protein associated with a carrier such that the antigen presenting cells (e.g., dendritic cells) can be pre-activated for uptake of the recombinant protein as antigens.

The inventors further contemplate that recombinant proteins (e.g., with carrier or without carrier) or cancer vaccines producing the recombinant protein can be used to produce T cells specific to antigen presenting cells presenting the neoepitope and calreticulin. For example, isolated dendritic cells (e.g., patient's own dendritic cells derived from the patient's blood, or immortalized human dendritic cell lines, etc.) can be exposed to the cancer vaccines (virus, bacteria, yeast vaccines, etc.) or the recombinant protein (with or without an adjuvant) such that the dendritic cells can process the recombinant protein as an antigen to so present at least a portion of calreticulin and the tumor associated antigen on the cell surface. Then, immune competent cells (e.g., CD4+ T cells, CD8+ T cells, NK cells, NKT cells, or combination of any of those) can contact the dendritic cells to be activated and develop specificity to calreticulin and the tumor associated antigen. In such example, it is especially preferred that the immune competent cells are derived and/or isolated from the patient, and optionally expanded ex vivo, to reduce or avoid potential allograft rejection. Then, the activated immune competent cells can be administered to the patient systemically or intratumorally.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Where the specification claims refers to at least one of something selected from the group consisting of A, B, C . . . and N, the text should be interpreted as requiring only one element from the group, not A plus N, or B plus N, etc.

What is claimed is:

1. A recombinant expression vector for immune therapy, comprising:
a nucleic acid sequence that encode a recombinant protein;
wherein nucleic acid sequence comprises a first nucleic acid segment encoding at least a portion of calreticulin and a second nucleic acid segment encoding at least one of a portion of a transmembrane domain or a tumor associated antigen; and
wherein the first and second nucleic acid segments are present in a same reading frame.

2. The expression vector of claim 1, wherein the second nucleic acid segment encodes a portion of a transmembrane domain, and the nucleic acid sequence further comprises a third nucleic acid segment encoding a tumor associated antigen.

3. The expression vector of claim 1, wherein the expression vector is selected from a group consisting of a viral expression vector, a bacteria expression vector, and a yeast expression vector.

4. The expression vector of claim 1, wherein the portion of the calreticulin is a mutant form of calreticulin, in which KDEL sequence is deleted from carboxyl terminus.

5. The expression vector of claim 1, wherein the second nucleic acid segment is coupled with the first nucleic acid segment via a linker.

6. The expression vector of claim 1, wherein the portion of the calreticulin is coupled with a membrane targeting signal sequence.

7. The expression vector of claim 1, wherein the tumor associated antigen is a patient- and tumor-specific neoepitope.

8. The expression vector of claim 1, wherein the tumor associated antigen is a polytope that comprises a plurality of filtered neoepitope peptides.

9. The expression vector of claim 8, wherein the neoepitope is filtered to have binding affinity to an MHC-I or MHC-II complex of equal or less than 500 nM.

10. The expression vector of claim 8, wherein the neoepitope is filtered against known human SNP and somatic variations.

11. A method of increasing effectiveness of immune therapy to a patient having a tumor, comprising:
providing a pharmaceutical composition comprising a nucleic acid sequence that encodes a recombinant protein;
wherein nucleic acid sequence comprises a first nucleic acid segment encoding at least a portion of calreticulin and a second nucleic acid segment encoding at least one of a portion of a transmembrane domain or a tumor associated antigen;
wherein the first and second nucleic acid segment are present in a same reading frame; and
administering the pharmaceutical composition to the patient in a dose and schedule effective to treat the tumor.

12. The method of claim 11, wherein the portion of the calreticulin is a mutant form of calreticulin, in which KDEL sequence is deleted from carboxyl terminus.

13. The method of claim 11, wherein the second nucleic acid segment is coupled with the first nucleic acid segment via a linker.

14. The method of claim 11, wherein the portion of the calreticulin is coupled with a membrane targeting signal sequence.

15. The method of claim 11, wherein the second nucleic acid segment encodes a portion of a transmembrane domain, and the nucleic acid sequence further comprises a third nucleic acid segment encoding a tumor associated antigen.

16. A pharmaceutical composition comprising:
a recombinant peptide comprising at least a portion of calreticulin and a tumor associated antigen; and
an adjuvant molecule stimulating a dendritic cell.

17. The composition of claim 16, wherein at least one of the recombinant peptide and the adjuvant molecule are coupled with a molecular carrier.

18. The composition of any one of the claims 58-60, wherein the tumor associated antigen is a patient- and tumor-specific neoepitope.

19. The composition of claim 61, wherein the neoepitope is filtered to have binding affinity to an MHC-I or MHC-II complex of equal or less than 500 nM or filtered against known human SNP and somatic variations.

20. The composition of any one of the claims 58-65, wherein the adjuvant molecule is Bacillus Calmette-Guerin (BCG).


SEQUENCE LISTING (6 sequences)

SEQ ID NO: 1 - protein, 4 aa, Artificial Sequence
  ER retention signal sequence
KDEL

SEQ ID NO: 2 - protein, 4 aa, Artificial Sequence
  synthetic sequence
GGGG

SEQ ID NO: 3 - protein, 6 aa, Artificial Sequence
  synthetic sequence
GGGGGG

SEQ ID NO: 4 - protein, 4 aa, Artificial Sequence
  synthetic sequence
GGEL

SEQ ID NO: 5 - protein, 4 aa, Artificial Sequence
  synthetic sequence
GDGL

SEQ ID NO: 6 - protein, 5 aa, Artificial Sequence
  synthetic sequence
GGSGG